(19) United States
(12) Patent Application Publication
(10) Pub. No.: US 2008/0300460 A1
(43) Pub. Date: Dec. 4, 2008

(54) DISTAL END PORTION OF ENDOSCOPE

(75) Inventor: Noriyuki SUGITA, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/118,945

(22) Filed: May 12, 2008

(30) Foreign Application Priority Data
May 28, 2007 (JP) 2007-139974

(51) Int. Cl. A61B 1/00

(52) U.S. Cl. 600/127

(57) ABSTRACT

A distal end portion of an endoscope includes a distal end portion body provided to a distal end of an insertion part of the endoscope, and a hood member configured to be projected in a shape of cylinder forward from an entire outer circumferential portion of the distal end portion body, which hood member includes an accordion-like portion configured to be stretched and shrunken in an axis line direction thereof and keep a desired length thereof.

### BACKGROUND OF THE INVENTION

The following description relates to one or more distal end portions of an endoscope.

In endoscopes, in particular, forward-viewing endoscopes, there are many ones having a hood member, which is projected in a shape of a cylinder forward from an entire circumferential portion of a distal end portion body provided at a leading edge of an insertion part of the endoscope. A main reason why the hood member is provided to the endoscope is, for example, to secure an observation distance up to an observed subject in a hollow viscus such as an esophagus and large intestine. A desired one of various hood members with a straight distal end or obliquely-cut distal end is selectively used depending on a purpose.

A projected length of the hood member has to be changed depending on a condition of an observed region or an intended use of the endoscope (for example, for normal observation or treatment, or for what kind of treatment). As an endoscope to meet this requirement, there has been known one with a hood member configured to be slidable back and forth with respect to a main body of a distal end portion in accordance with an operation under a user's hand (for example, see Japanese Patent Provisional Publication No. 2000-79086, which is hereinafter referred to as '086 Publication).

However, a configuration of the distal end portion with the hood member results in a greater diameter of a distal end of the insertion part and thus a greater pain given to a person being examined by the endoscope. Hence, since the endoscope disclosed in '086 Publication needs a further mechanism to attain the aforementioned configuration of the distal end portion with the slidable hood member, it causes a further increased diameter of the distal end portion and thus a further increased pain that the person being examined suffers. Further, it is impossible to change the direction of a distal end of the hood member.

### SUMMARY OF THE INVENTION

The present invention is advantageous in that there is provided an improved distal end portion of an endoscope that makes it possible to easily adjust a projected length of a hood member without having to increase an outer diameter of a distal end of an insertion part, and further to easily adjust a direction of a distal end of the hood member.

According to an aspect of the present invention, there is provided a distal end portion of an endoscope, which includes a distal end portion body provided to a distal end of an insertion part of the endoscope, and a hood member configured to be projected in a shape of cylinder forward from an outer circumferential portion of the distal end portion body, the hood member including an accordion-like portion configured to be stretched and shrunken in an axis line direction thereof.

Optionally, the hood member may be configured to keep a desired length thereof in the axis line direction.

Optionally, the hood member may detachably be fixed to the outer circumferential portion of the distal end portion body with an adhesive tape wound around a rear end portion of the hood member.

Still optionally, the accordion-like portion may be formed at a portion of the hood member except for a front end portion thereof.

Further optionally, the hood member may be bendable at the accordion-like portion and capable of keeping a bent state.

Yet optionally, in a state where the accordion-like portion is the most stretched in the axis line direction, a distance from a distal end face of the distal end portion body to a front end surface of the hood member may be equal to or larger than twice an outer diameter of the distal end portion body.

Optionally, the hood member may include a front end surface formed to be oblique with respect to a distal end face of the distal end portion body in a state where the accordion-like portion is completely compressed and shrunken.

Optionally, an outer diameter of a portion of a hood member at which the accordion-like portion is formed may be equal to or smaller than an outer diameter of a portion of the hood member at which the accordion-like portion is not formed.

Optionally, the accordion-like portion of the hood member may be formed around an outer circumferential surface of the distal end portion body.

Further optionally, when the accordion-like portion is shrunken, a front end surface of the hood member may conform to the distal end face of the distal end portion body.

### BRIEF DESCRIPTION OF THE ACCOMPANYING DRAWINGS

FIG. 1 is a perspective view of a distal end portion of an endoscope in a first embodiment according to one or more aspects of the present invention.

FIG. 2 is a cross-sectional side view of the distal end portion of the endoscope in the first embodiment according to one or more aspects of the present invention.

FIG. 3 is a cross-sectional side view of the distal end portion of the endoscope in a state where a hood member is the most stretched in the first embodiment according to one or more aspects of the present invention.

FIG. 4 is a cross-sectional side view of the distal end portion of the endoscope in a state where the hood member is intermediately stretched in the first embodiment according to one or more aspects of the present invention.

FIG. 5 is a cross-sectional side view of the distal end portion of the endoscope in a first usage example in the first embodiment according to one or more aspects of the present invention.

FIG. 6 is a cross-sectional side view of the distal end portion of the endoscope in a second usage example in the first embodiment according to one or more aspects of the present invention.

FIG. 7 is a cross-sectional side view of the distal end portion of the endoscope in a state where the hood member is bent at an accordion-like portion in the first embodiment according to one or more aspects of the present invention.

FIG. 8 is a cross-sectional side view of the distal end portion of the endoscope in an example used in a state where the hood member is bent at an accordion-like portion in the first embodiment according to one or more aspects of the present invention.

FIG. 9 is a cross-sectional side view of the distal end portion of the endoscope for defining dimensions of the distal end portion in the first embodiment according to one or more aspects of the present invention.

FIG. 10 is a cross-sectional side view of a distal end portion of the endoscope in a second embodiment according to one or more aspects of the present invention.

FIG. 11 is a cross-sectional side view of the distal end portion of the endoscope in a state where a hood member is intermediately bent in the second embodiment according to one or more aspects of the present invention.

FIG. 12 is a cross-sectional side view of the distal end portion of the endoscope in a state where the hood member is the most bent in the second embodiment according to one or more aspects of the present invention.

FIG. 13 is a cross-sectional side view of a distal end portion of the endoscope in a third embodiment according to one or more aspects of the present invention.

FIG. 14 is a cross-sectional side view of a distal end portion of the endoscope in a fourth embodiment according to one or more aspects of the present invention.

FIG. 15 is a cross-sectional side view of the distal end portion of the endoscope in a state where a hood member is intermediately stretched in the fourth embodiment according to one or more aspects of the present invention.

### DETAILED DESCRIPTION OF THE EMBODIMENTS

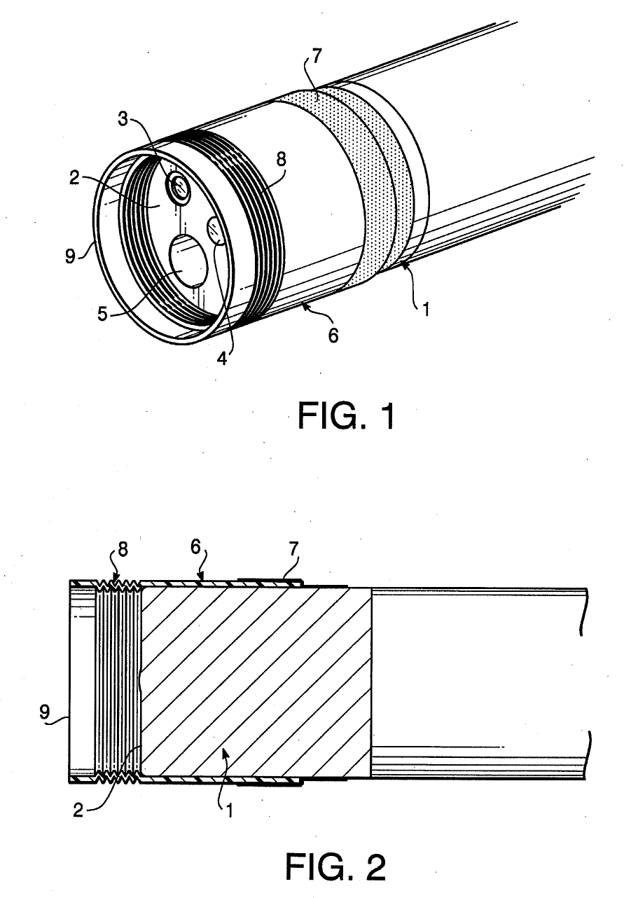

Hereinafter, embodiments according to aspects of the present invention will be described with reference to the accompanying drawings. FIG. 1 is a perspective view of a distal end portion of an endoscope in a first embodiment. FIG. 2 is a cross-sectional side view of the distal end portion of the endoscope. At a distal end face 2 of a distal end portion body 1 joined to a leading edge of a flexible insertion part, there are provided an observation port 3 for viewing in front thereof, illumination port 4, and treatment tool outlet port 5. The treatment tool outlet port 5 is an outlet opening of a treatment tool insertion channel (not shown) inserted into the insertion part, and doubles as a suction port.

There is detachably attached to the distal end portion body 1, a cylinder-shaped hood member 6 projected forward from an entire outer circumferential portion of the distal end portion body 1. In the present embodiment, the hood member 6 is formed to have such an inner diameter that the outer circumference of the distal end portion body 1 is fitted thereinto. Further, the hood member 6 is detachably fixed to the outer circumferential portion of the distal end portion body 1 with an adhesive tape 7 wound around both an outer circumference of a rear end of the hood member 6 and the outer circumferential surface of the distal end portion body 1.

Accordingly, by unsticking the adhesive tape 7, the hood member 6 can be detached from the distal end portion body 1. It is noted that there may be employed to detachably fix the hood member 6 to the distal end portion body 1, any of already-known configurations such as a hood member 6 formed from resilient material so as to fasten the outer circumferential portion of the distal end portion body 1, and engagement between a concavity and convexity provided at the inner circumference of the hood member 6 and the outer circumference of the distal end portion body 1. In addition, the hood member 6 may be configured so as not to be detached.

The hood member 6 is provided with an accordion-like portion 8 formed, for example, from polyethylene resin plastic material, which accordion-like portion 8 is elastic in an axis line direction and capable of keeping any length thereof in the axis line direction. The capability of the accordion-like portion 8 to keep the length thereof in the axis line direction is dependent on a property thereof. The length of the accordion-like portion 8 in the axial direction cannot be changed by a compressive or tensile force up to some extent, yet can be changed by more than a predetermined compressive or tensile force.

In the present embodiment, the accordion-like portion 8 is not formed at a portion into which the distal end portion body 1 is fitted. Further, a rear end of the accordion-like portion 8 is located in the vicinity of the distal end face 2 of the distal end portion body 1 while a front end thereof is spaced from a front end surface 9 of the hood member 6. Thus, since the accordion-like portion 8 is not formed in the vicinity of the front end surface 9 of the hood member 6, an observation viewing field is not narrowed.

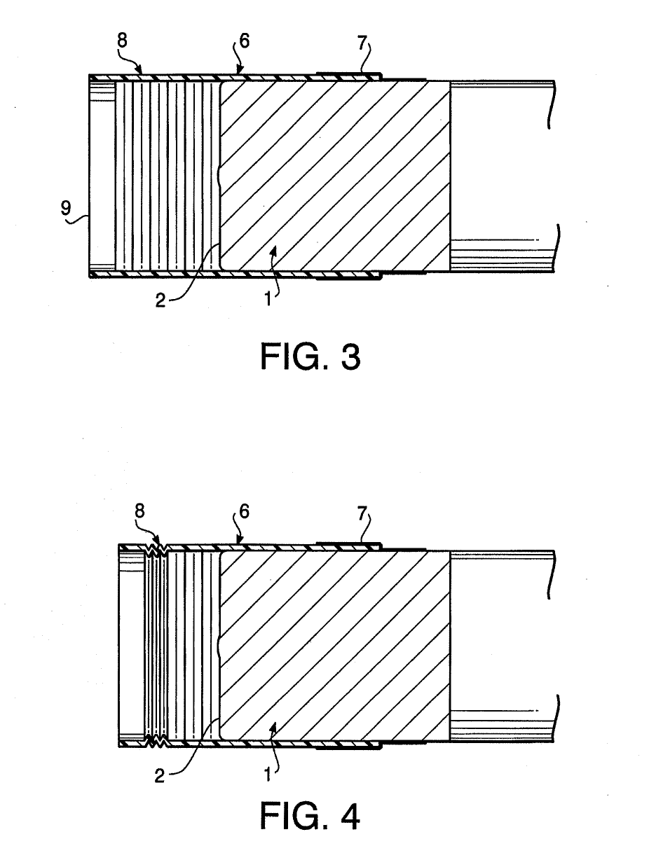

When being pulled forward, the hood member 6 configured as above can be long stretched in the axis line direction as shown in FIG. 3 from a state where the accordion-like portion 8 is the most shrunken as shown in FIG. 2. FIG. 3 shows the longest state of the hood member 6 in the axis line direction.

In addition, as shown in FIG. 4, when the accordion-like portion 8 is set in a required intermediate state between the most shrunken state and the most stretched state, the hood member 6 is set and kept in a state of a required length in the axis line direction between the shortest state and the longest state.

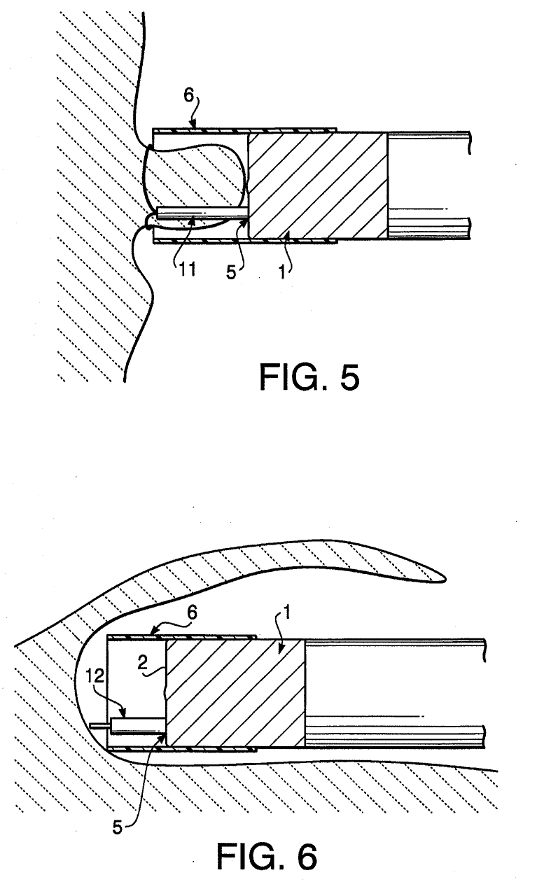

Hence, for example, as shown in FIG. 5, when there is performed a treatment of constricting living tissue with a high-frequency snare 11 projected out of the treatment tool outlet port 5, a lot of living tissue can be taken at a time with the hood member set in the longest state.

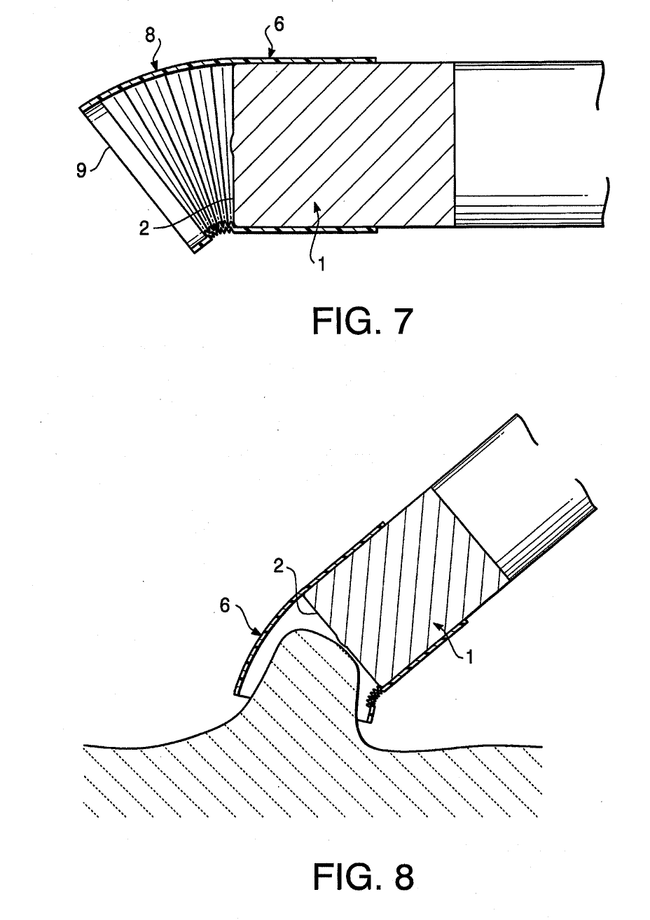

Further, for example, as shown in FIG. 6, when there is performed a mucosa peeling treatment with a high-frequency knife 12, the hood member 6 set in a somewhat shorter state is more convenient since the high-frequency knife 12 is less wobbly.

The hood member 6 formed with the accordion-like portion 8 can not only be stretched and shrunken in the axis line direction, but as well be bent at the accordion-like portion 8 and kept in the bent state against an external force less than a predetermined force, as shown in FIG. 7.

Thus, for example, as shown in FIG. 8, even though a treatment subject is living tissue at such a region as not to face the distal end face 2 of the distal end portion body 1, the front end surface 9 of the hood member 6 can be directed to face such living tissue by setting the hood member 6 in a required bent state. Therefore, the living tissue as a treatment subject can be sucked into the hood member 6 by suction from the treatment tool outlet port 5 which doubles as a suction port, and thereby a treatment with the high-frequency snare 11 can easily be done in the same manner as shown in FIG. 5.

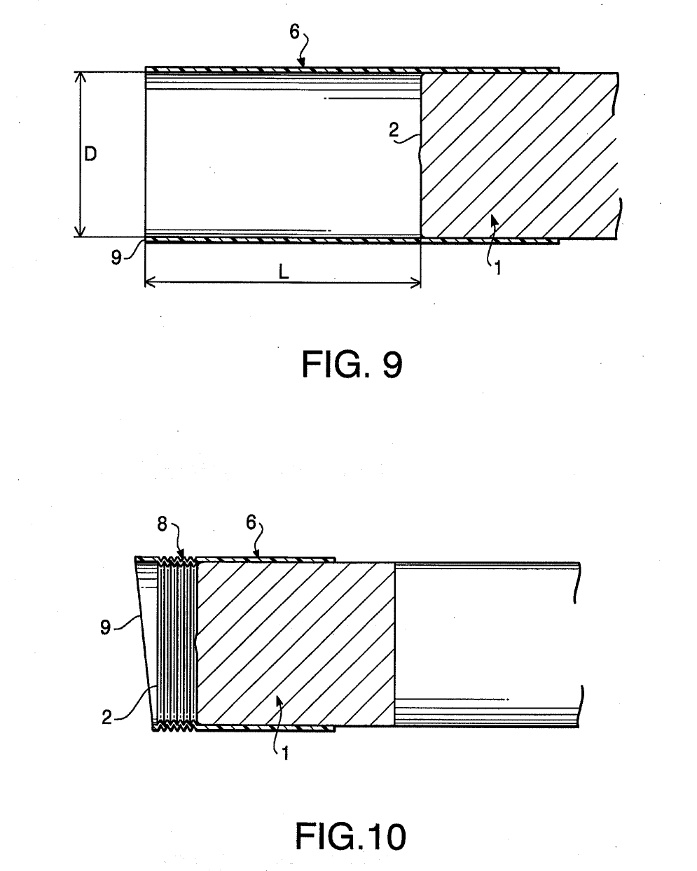

It is noted that, in order to perform such a treatment as the user likes, as shown in FIG. 9, the accordion-like portion 8 is desired to be formed, in the most stretched state in the axis line direction, such that a length L from the distal end face 2 of the distal end portion body 1 to the front end surface 9 of the hood member 6 is equal to or larger than twice an outer diameter D of the distal end portion body 1 (viz., L≧2 D).

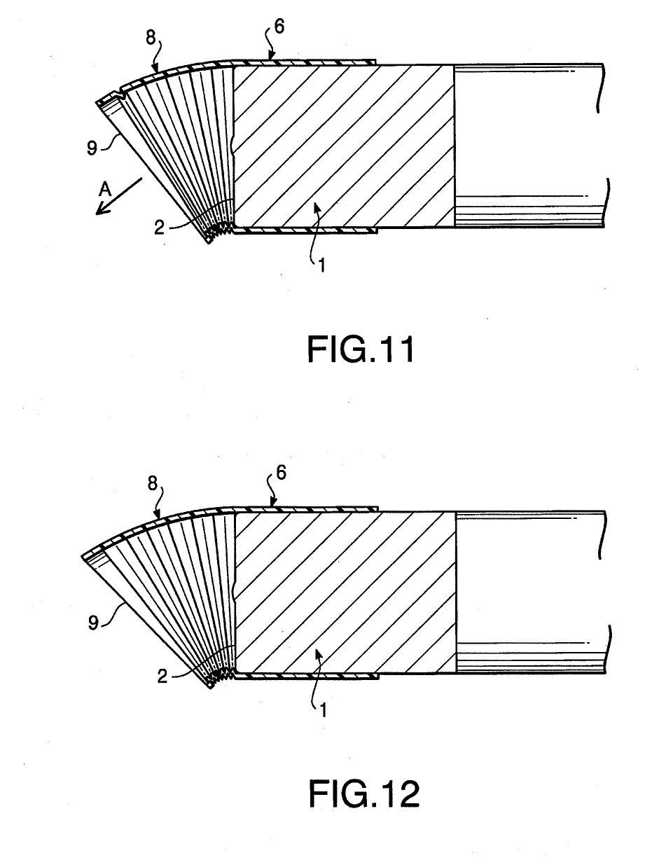

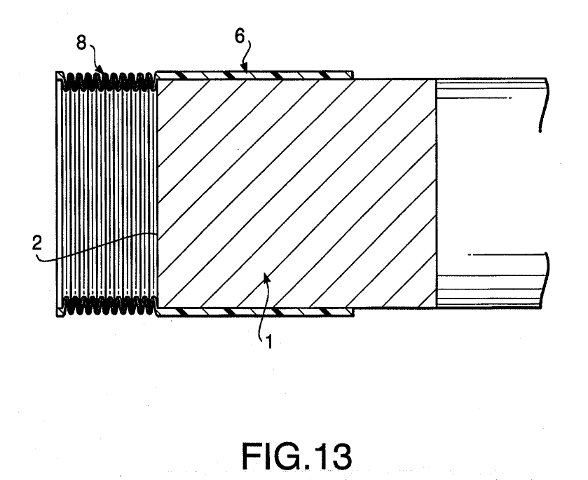

FIG. 10 is a cross-sectional side view of a distal end portion of the endoscope in a second embodiment according to aspects of the present invention. As shown in FIG. 10, in the second embodiment, a front end surface 9 of a hood member 6 is formed to be oblique with respect to the distal end face 2 of the distal end portion body 1 in a state where the accordion-like portion 8 is completely compressed and shrunken. In the second embodiment, by pulling the hood member 6 in an oblique direction as indicated by an arrow A in FIG. 11, the front end surface 9 of the hood member 6 can be set in a largely oblique state as shown in FIG. 12.

FIG. 13 is a cross-sectional side view of a distal end portion of the endoscope in a third embodiment according to aspects of the present invention. As shown in FIG. 13, in the third embodiment, folded portions of an accordion-like portion 8 are formed to be protruded inward in a radial direction such that an outer diameter of a portion of a hood member 6 at which the accordion-like portion 8 is formed is not greater than an outer diameter of a portion of the hood member 6 at which the accordion-like portion 8 is not formed. Even though the hood member 6 is provided with the accordion-like portion 8, the aforementioned configuration can attain a small outer diameter of the hood member 6 and thus an easy inserting operation of the distal end portion.

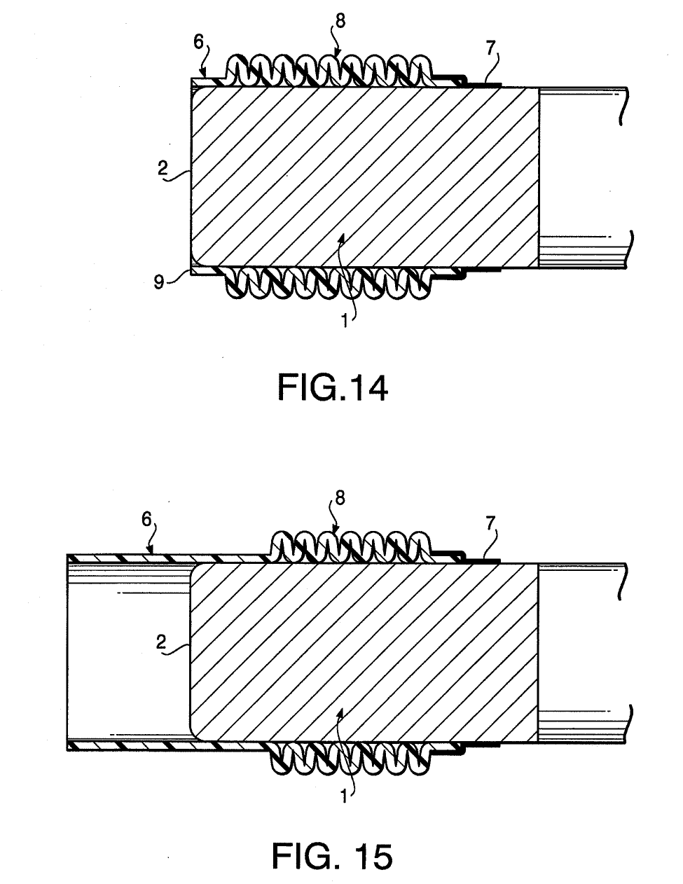

FIG. 14 is a cross-sectional side view of a distal end portion of the endoscope in a fourth embodiment according to aspects of the present invention. As shown in FIG. 14, in the fourth embodiment, an accordion-like portion 8 of a hood member 6 is formed only around the outer circumferential surface of the distal end portion body 1. Therefore, it is possible to conform a front end surface 9 of the hood member 6 to the distal end face 2 of the distal end portion body 1, and thus attain an easy inserting operation of the distal end portion.

According to the configuration, by pulling the hood member 6 in the axis line direction, as shown in FIG. 15, the accordion-like portion 8 is stretched, and the hood member 6 is protruded in front of the distal end portion body 1. Thus, the fourth embodiment can as well provide the same effects as the first embodiment.

The present disclosure relates to the subject matter contained in Japanese Patent Application No. P2007-139974, filed on May 28, 2007, which is expressly incorporated herein by reference in its entirety.

What is claimed is:

1. A distal end portion of an endoscope, comprising:
a distal end portion body provided to a distal end of an insertion part of the endoscope; and
a hood member configured to be projected in a shape of cylinder forward from an outer circumferential portion of the distal end portion body,
the hood member including an accordion-like portion configured to be stretched and shrunken in an axis line direction thereof and keep a desired length thereof.

2. The distal end portion of the endoscope according to claim 1,
wherein the hood member is configured to keep a desired length thereof in the axis line direction.

3. The distal end portion of the endoscope according to claim 1,
wherein the hood member is detachably fixed to the outer circumferential portion of the distal end portion body with an adhesive tape wound around a rear end portion of the hood member.

4. The distal end portion of the endoscope according to claim 1,
wherein the accordion-like portion is formed at a portion of the hood member except for a front end portion thereof.

5. The distal end portion of the endoscope according to claim 1,
wherein the hood member is bendable at the accordion-like portion and capable of keeping a bent state.

6. The distal end portion of the endoscope according to claim 1,
wherein, in a state where the accordion-like portion is the most stretched in the axis line direction, a distance from a distal end face of the distal end portion body to a front end surface of the hood member is equal to or larger than twice an outer diameter of the distal end portion body.

7. The distal end portion of the endoscope according to claim 1,
wherein the hood member includes a front end surface formed to be oblique with respect to a distal end face of the distal end portion body in a state where the accordion-like portion is completely compressed and shrunken.

8. The distal end portion of the endoscope according to claim 1,
wherein an outer diameter of a portion of a hood member at which the accordion-like portion is formed is equal to or smaller than an outer diameter of a portion of the hood member at which the accordion-like portion is not formed.

9. The distal end portion of the endoscope according to claim 1,
wherein the accordion-like portion of the hood member is formed around an outer circumferential surface of the distal end portion body.

10. The distal end portion of the endoscope according to claim 9,
wherein, when the accordion-like portion is shrunken, a front end surface of the hood member conforms to the distal end face of the distal end portion body.
